(19) United States
(12) Patent Application Publication
(10) Pub. No.: US 2022/0087584 A1
(43) Pub. Date: Mar. 24, 2022

(54) DECEPTION DETECTION USING OCULOMOTOR, CARDIOVASCULAR, RESPIRATORY AND ELECTRODERMAL MEASURES

(71) Applicant: Converus, Inc., Lehi, UT (US)

(72) Inventors: John C. Kircher, Lehi, UT (US); Mark D. Handler, Lehi, UT (US); Grant G. Parkinson, Lehi, UT (US); Benjamin C. Stout, Lehi, UT (US)

(73) Assignee: Converus, Inc., Lehi, UT (US)

(21) Appl. No.: 17/330,294

(22) Filed: May 25, 2021

Related U.S. Application Data
(60) Provisional application No. 63/082,345, filed on Sep. 23, 2020.

(51) Int. Cl. A61B 5/16; A61B 5/0533; A61B 5/0205; A61B 5/00

(52) U.S. Cl. CPC A61B 5/164; A61B 5/163; A61B 5/165; A61B 2562/22; A61B 5/0205; A61B 5/7264; A61B 5/7289; A61B 5/0533

(57) ABSTRACT

The present application discloses a deception detection systems and processes that use a processor; an eye tracking device connected to the processor, and a set of physiological sensors connected to the processor. The eye tracking device may comprise an infrared camera that is positioned to be able to track the oculomotor activity of a subject. The deception detection system also includes a respiratory sensor connected to the processor positioned to be able to track the respiration of the subject and an electrodermal sensor connected to the processor positioned to be able to detect the electrodermal activity of the subject. Additionally, the deception detection system includes a cardiovascular sensor connected to the processor positioned to be able to detect the cardiovascular activity of the subject and a speaker connected to the processor positioned to be able to transmit sound to the subject.

### CROSS-REFERENCE TO RELATED APPLICATION

This application claims the benefit of U.S. Provisional Application No. 63/082,345, filed Sep. 23, 2020, which is incorporated herein by reference in its entirety, including but not limited to those portions that specifically appear hereinafter.

### TECHNICAL FIELD

This invention relates to automated deception detection testing methods, systems, and protocols.

### BACKGROUND

U.S. Publication Patent No. 2010/0324454, the contents of which are incorporated herein by reference, discloses rapid, automated methods for using oculomotor measures to decide whether a person is being truthful or deceitful. The original oculomotor deception test (ODT) was described in a doctoral dissertation by D. Osher in 2006. Since then, various ODT test formats have been developed that build on the design of the original ODT. These include the relevant comparison test (RCT), the multiple-issue comparison test (MCT), an audio version of the multiple comparison test (AMCT) and the ocular directed lie comparison test (DLC). In the present disclosure, the term ODT encompasses all such derivative ocular-based test formats. Commercial products embodying such methods have been released under the brand name EyeDetect and have proven successful in the marketplace. However, various federal and state laws require other physiological measures be used by a lie detector to meet the requirements of the Employee Polygraph Protection Act of 1988 and several state laws. Expansion of EyeDetect to include traditional polygraph channels would meet those requirements and increase the usefulness and applicability of the technology.

Because it would include both oculomotor and traditional polygraph measures, such a system should have increased accuracy in comparison to prior art systems. A hybrid system that combines not only polygraph and oculomotor measures but also polygraph and ODT interrogation techniques would be another improvement in the art.

### SUMMARY

The present disclosure is an automated method for deception detection that combines polygraph and oculomotor deception detection technologies, both of which have been shown to achieve greater than chance levels of accuracy. Both types of tests base inferences about truth and deception on within-subject comparisons of reactions to different types of test questions. However, the polygraph is, in part, an emotion-based test that assumes some questions cause reactions because they are more threatening than others, whereas the ODT is primarily a cognition-based test that assumes that it is cognitively more demanding to lie to some questions than to tell the truth to others. In the present disclosure, traditional polygraph and ODT tests are administered sequentially to obtain emotional and cognitive assessments of the test subject's veracity. In one embodiment, the first phase of the test is an automated polygraph test, with inter-question intervals that are optimized to provide traditional polygraph data that are diagnostic of deception. Traditional polygraph signals include respiration movement-related activity, electrodermal activity, and cardiovascular activity. Those measures are particularly sensitive to changes in autonomic and somatic arousal associated with deception. In some embodiments, the polygraph phase of the test may be administered first because emotional reactions habituate rapidly and lose their ability to discriminate between truth and deception over the course of the test. The test may include a second phase that is optimized to provide oculomotor and behavioral measures of deception such as pupil size and eye movements, response time and wrong answers. Such measures are especially sensitive to changes in cognitive load and do not habituate appreciably over the course of the test. In one embodiment, the first and second phases of the test may be scored separately, and a posterior probability of deception derived from analysis of the polygraph data collected in the first phase may serve as a prior probability of deception in an analysis of the oculomotor and behavioral data from the second phase.

In another embodiment, features extracted from the polygraph recordings during the first phase may be combined with features extracted from the second phase to compute a single posterior probability of deception. The accuracy of the hybrid system should exceed that of prior art because the polygraph and ODT tests are based on different psychological processes and provide partially independent sources of diagnostic information about a person's deceptive status. Accuracy of the hybrid system also should improve because it includes more sources of diagnostic information than either the polygraph or oculomotor test in isolation. The present disclosure includes systems for conducting the testing protocols discussed herein, as well as the computer implemented methods related to such protocols.

In one illustrative embodiment, a system in accordance with the present disclosure may include a test station for presenting the instructions and questions to a test subject and capturing physiological and behavioral data for analysis. Such a test station may include a computer that presents a series of questions about illicit behaviors, a physiological recorder that collects respiration, electrodermal, cardiovascular, and somatic activity along with a remote eye- tracker that uses an infrared camera to record eye movements and pupil size, and a chin rest. The computer may present test instructions and questions as text on the computer monitor and/or aurally with a computer-synthesized voice.

Systems, methods and procedures in accordance with the present disclosure are improvements over traditional polygraph testing because they replace the polygraph examiner with a computer for the administration and analysis of the test data. Polygraph examiners vary from day to day and from one examiner to another. Variance in the manner the polygraph test is administered and scored can affect the test outcomes. In contrast, in a completely standardized manner, a computer can describe the rationale underlying the polygraph test to the test subject, conduct a pretest review of the topics covered on the test, present instructions, provide practice and feedback to the test subject, and present test questions temporally spaced to optimize the diagnostic information available in respiration, electrodermal, and cardiovascular measures.

In contrast to a human examiner, a system in accordance with the present disclosure also can extract many diagnostic features from respiration, electrodermal and cardiovascular signals, gaze position, and pupil size and can do so in consistent manner in a matter of seconds. For traditional polygraph physiological measures, the most diagnostic features are amplitude of the electrodermal reactions, baseline increases in relative blood pressure, and suppression of respiration as measured by the length of the respiration tracing for a fixed period of time starting at question onset. Given a set of diagnostic features, logistic regression analysis may be used to combine them in a mathematically optimal manner to discriminate between truthful and deceptive individuals. The obtained regression equation may weigh features according to their diagnosticity and statistical independence and output the probability of deception. A probability cutoff then may be used to classify test subjects as deceptive or credible. For example, if the probability of deception exceeds a cutoff of 0.5, then classify the test subject as deceptive. If the probability is less than 0.5, then classify the test subject as credible. Two cutoffs may be used to establish an inconclusive region. For example, if the probability of deception is between 0.4 and 0.6, then the test is inconclusive. Otherwise, classify the test subject as deceptive if the probability exceeds 0.6 or credible if the probability of deception falls below 0.4.

For traditional polygraph tests, it is important to allow physiological reactions to test questions sufficient time to recover before the next question is presented. Strong reactions to a question in a polygraph examination can require 20 or more seconds to recover. Accordingly, hybrid systems in accordance with the present disclosure may impose an inter-question interval of 20 seconds or more during the polygraph phase of a test. Additionally, by using a protocol that never places two questions that address the same relevant issue in succession, any reactions that carry over to the next question should have additional time to recover.

In one illustrative embodiment of an examination conducted in accordance with the present disclosure, the system may present a series of 10 to 12 questions three times and if the outcome is inconclusive, the questions may be presented another two times. In one illustrative embodiment of the present disclosure, the test in accordance with the present disclosure may cover four relevant issues. Four questions ask about each of the four relevant issues for a total of 16 unique questions, and the set of 16 questions is presented twice. The order of question presentation may be randomized subject to the constraint that no two questions that address the same relevant issue are presented in succession. This provides eight opportunities to measure respiratory, electrodermal, cardiovascular, and oculomotor reactions to questions concerning each of four relevant topics. In some such embodiments, the questions may be arranged in all possible pairs of the relevant issues. In the first half of a first session, questions about the first relevant issue (R1) may be paired with questions about the second relevant issue (R2). In a second half of the first session, questions about the third relevant issue (R3) may be paired with questions about the fourth relevant issue (R4). In subsequent sessions, the first relevant issue (R1) may be paired with the third relevant issue (R3), the first relevant issue (R1) may be paired with the fourth relevant issue (R4), the second relevant issue (R2) may be paired with the third relevant issue (R3), and the second relevant issue (R2) may be paired with the fourth relevant issue (R4). During any given sub-session, the questions focus on only two of the four relevant issues.

Unlike a traditional polygraph examination, where often a polygraph examiner reads each test question aloud and the test subject verbally answers “Yes” or “No”, embodiments in accordance with the present disclosure may present test questions aurally with computer synthesized voice over noise-cancelling headphones and have the test subject answer “Yes” or “No” by pressing the left or right mouse button.

The testing station may record oculomotor, respiratory, electrodermal, and cardiovascular changes, as well as response times and wrong answers. At the conclusion of the test, the testing station may extract diagnostic features from the various physiological channels and behavioral measures. In one embodiment, the polygraph and ODT data may be scored separately. Logistic regression may be used to compute the posterior probability of deception based on either polygraph physiological data or the ODT data. A mathematical equation may be used to adjust the probability of deception from the polygraph phase based on a logistic regression analysis of data collected during the ODT phase. A final posterior probability of deception based on both sets of data may be used to classify the test subject as credible or deceptive. In another embodiment, a single logistic regression equation may be generated using features extracted from the polygraph and ODT measurements to compute a single posterior probability of deception based on both phases of the hybrid test.

This application further discloses a process comprising a processor that is transmitting, through a speaker, a question aurally to a subject to obtain a cognitive assessment and transmitting, by the one or more processors, through the speaker, a question aurally to a subject to obtain an emotional assessment. The processor is further receiving oculomotor movement of the subject from an eye tracking device that includes an infrared camera and receiving a pupil size of a subject from the eye tracking device. Additionally, the processor is receiving respiration of the subject from a respiratory sensor and cardiovascular activity of the subject from a cardiovascular sensor. Moreover, the processor is receiving, by electrodermal activity of the subject from an electrodermal sensor. This in turn allows calculating, by the one or more processors, a cognitive assessment and an emotional assessment using the physiological measurements of the subject. The processor is later used in determining the credibility score of the subject using both the cognitive assessment and the emotional assessment; and displaying the credibility score on a screen.

### DESCRIPTION OF THE DRAWINGS

It will be appreciated by those of ordinary skill in the art that the various drawings are for illustrative purposes only. The nature of the present disclosure, as well as other embodiments in accordance with this disclosure, may be more clearly understood by reference to the following detailed description, to the appended claims, and to the several drawings.

FIGS. 1A and 1B depict one example of a set of physiological sensors that measure cardiovascular, respiratory, and electrodermal activity, which may be used in some illustrative systems and methods in accordance with the present disclosure.

FIG. 2 is an example of an image that may be presented on a visual display to monitor a test subject's oculomotor, cardiovascular, respiration and electrodermal activity during both phases of a testing protocol in accordance with the present disclosure.

FIG. 3 is another example of an image that may be presented on a visual display while data are collected from a test subject's oculomotor, cardiovascular, respiration and electrodermal activity during a testing protocol in accordance with the present disclosure.

FIG. 4 is an example of an image that may be presented on a visual display of the cardiovascular, respiration and electrodermal signals from a completed test using a testing protocol in accordance with the present disclosure.

FIG. 5 is an example of a report that may be generated by the system that shows the calculated test score from an analysis of a completed test using a testing protocol in accordance with the present disclosure.

### DETAILED DESCRIPTION

The present disclosure relates to apparatus, systems, and methods for computer-implemented deception detection testing. It will be appreciated by those skilled in the art that the embodiments herein described, while illustrative, are not intended to so limit this disclosure or the scope of the appended claims. Those skilled in the art will also understand that various combinations or modifications of the embodiments presented herein can be made without departing from the scope of this disclosure. All such alternate embodiments are within the scope of the present disclosure.

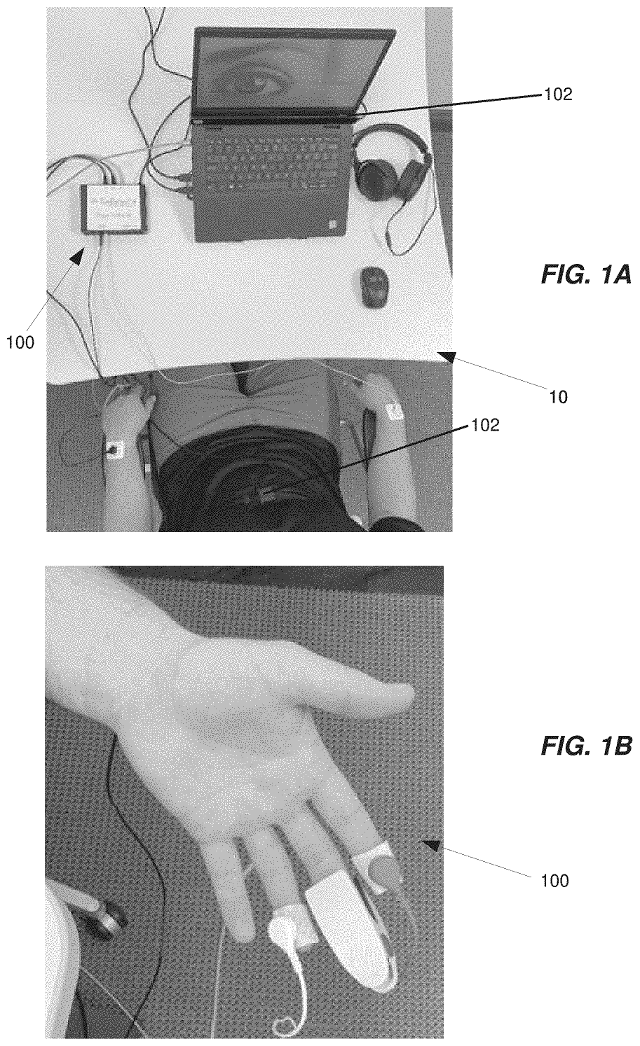

The present disclosure includes systems for conducting the testing protocols discussed herein, as well as the computer implemented methods related to such protocols. It will be appreciated that in some exemplary embodiments, such systems may include a test station for administering the tests to a test subject and capturing the oculomotor and other data for analysis. One exemplary test station 10, depicted in FIG. 1A may include a physiological recorder, an eye tracking device with an infrared camera 102, computer, keyboard and mouse, and a chin rest may be used. An audio output for speakers or noise-cancelling headphones may be included as a part of the test station. A set of pre-test instructions may be presented to the test subject in an audio format, as by text-to-speech narration software.

One example of a suitable test station is an EyeDetect+ Station which is commercially available from CONVERUS of Lehi, Utah. The primary hardware components of the EyeDetect+ Station include an eye tracking device (infrared camera) 102, a set of physiological sensors 100 that measure respiratory, electrodermal, cardiovascular, and body movement activity, a Windows-based computer, wireless keyboard and mouse and chin rest. Noise-cancelling headphones 104 are also included. The eye tracker is a high definition, infrared camera that takes up to 60 measurements per second of the examinee's eyes. Changes as small as 1/10th of a millimeter are detected. During a test, hundreds of thousands of eye measurements may be recorded, as well as answers to test questions and statements. The eye tracker has a tracking range of 32×21 cm at a distance of 60 cm from the test subject's eyes. It also has a +/−20° horizontal and +20°/−40° vertical range. During a test, eye measurements and the test subject's answers and response times are temporarily stored on an encrypted drive on the EyeDetect Station. Suitable systems and operating environments for doing the oculomotor data collection and analysis may also include those disclosed in [0056]-[0074] and FIGS. 1a and 1b of US 2010/0324454, as discussed previously herein, which systems include an audio output to present instructions, test items, and feedback to the test subject.

It will be appreciated that the set of physiological sensors 100 that measure respiration, electrodermal, cardiovascular and somatic activity may vary. One suitable version is depicted in FIGS. 1A and 1B. It includes two wires that attach to the fingers to measure electrodermal activity (EDA), two wires to measure the electrocardiogram (EKG), a photoelectric plethysmograph (PPG) sensor, a respiration strain gauge 104, and an activity monitor. Such sensors measure EDA, EKG, cardiovascular activity, blood volume pulse (BVP), heart rate, respiration-related movement, relative blood pressure changes and somatic activity. During a test, the station 100 continuously records electrodermal, cardiovascular, respiratory, and somatic activity, in addition to oculomotor and behavioral data. This allows the station and tests to satisfy the legal definition of lie detector as an “instrument that records continuously, visually, permanently, and simultaneously changes in cardiovascular, respiratory and electrodermal patterns”.

In measuring cardiovascular activity, the use of the EKG and PPG sensors allows a system in accordance with the present disclosure to measure and record a test subject's pulse transit time (PTT). This PTT data then can be used in the data analysis and determination associated with that test. Webb, Andrea K, USE OF PULSE TRANSIT TIME IN THE PSYCHOPHYSIOLOGICAL DETECTION OF DECEPTION, University of Utah 2006, available at Marriot Library General collection (HVI5.5 2006.W42), explains the suitability of PTT for such use and is incorporated by reference herein in its entirety. It will be appreciated that other embodiments that collect cardiovascular data in other manners, such as by use of a cardiograph to measure relative blood pressure may be used.

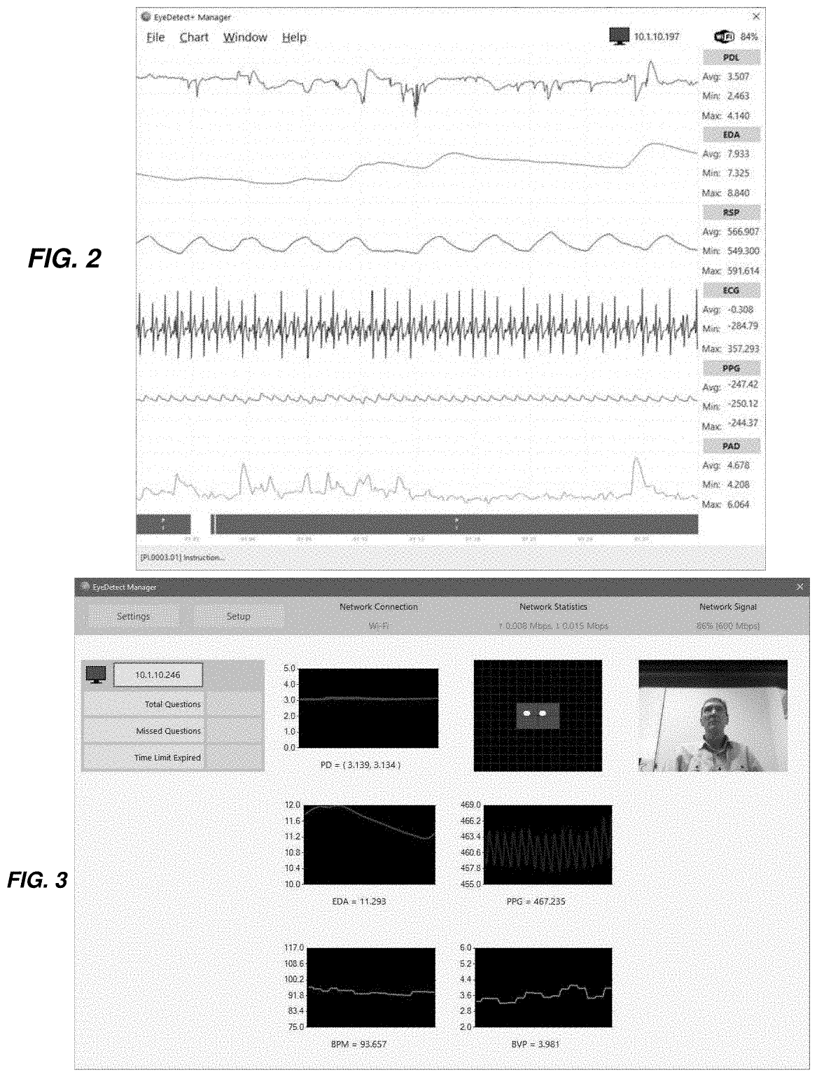

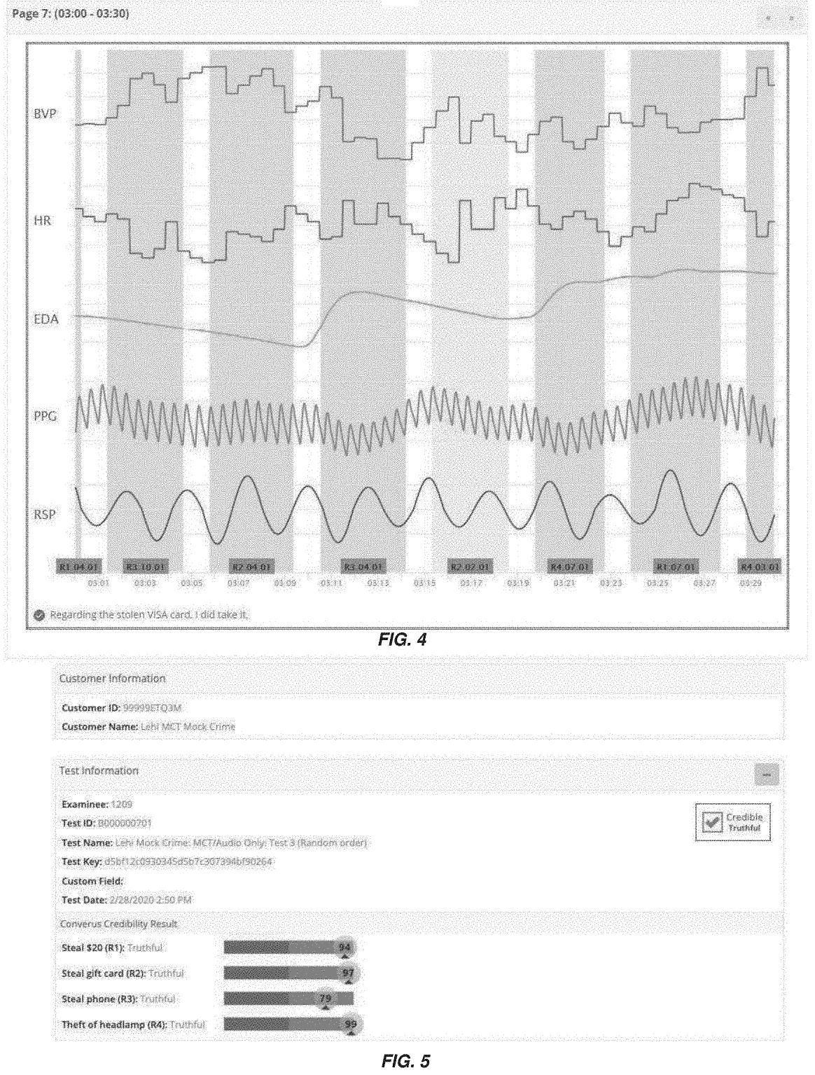

FIG. 2 depicts an image that visually displays the collection of the various data during a testing procedure that may be viewed by a test administrator. Such images may be generated by the system as a test subject takes the test and data are collected. Once a testing procedure is complete the data may be stored in a database and associated with a unique number to identify the test. FIGS. 3 and 4 depict images that visually display data collected during a testing procedure.

As discussed previously herein, in conducting a test in accordance with the present disclosure, the system may administer a test that measures a test subject's cognitive and emotional reactions to test questions to obtain a diagnostic value from traditional polygraph measures and to obtain a value from oculomotor measures. Such hybrid testing protocols assume deception is associated with increases in both cognitive effort and emotional arousal and that changes in recorded physiology load on one type of question or another, as a function of truth-telling or deception to the target issues. Although these assumptions have been confirmed many times in the scientific literature, to Applicant's knowledge, no one has developed a hybrid protocol that capitalizes on effects of deception on cognitive and emotional processes as indicated by oculomotor and traditional polygraph measures.

In one exemplary protocol, the system may administer a hybrid test that measures a test subject's cognitive and emotional reactions to test questions during an initial phase that optimizes the diagnostic value of traditional polygraph measures and during a second phase that optimizes the value of oculomotor measures. It will be appreciated that although the terms “first phase” and “second phase” are used, testing protocols directed to the generation and measurement of particular responses may be presented in differing order, or within a single session that mixes presentation type, dependent on the particular application.

The hybrid test is formatted to maximize the diagnostic value of oculomotor and other physiological measures. Polygraph test questions are presented aurally, answered “Yes” or “No” with a keypress or mouse click, and may be spaced 20 seconds apart to give physiological reactions sufficient time to recover before the next question is presented. Thus, traditional polygraph test questions may be presented at a rate of two or three questions a minute. In contrast, the oculomotor test questions are presented at a faster pace. To increase cognitive load effects on oculomotor measures, the computer instructs test subjects to answer the True/False statements as quickly and accurately as possible or they will fail the test. Depending on the individual, True/False statements typically are answered at a rate of 15 to 20 statements per minute. Oculomotor test statements are presented aurally or as text on a computer monitor and are answered “True” or “False” with a keypress or mouse click. The system presents the next statement half a second after the test subject answers. Oculomotor, respiratory, electrodermal, cardiovascular, and somatic activity are recorded continuously throughout both phases of the hybrid test. The computer extracts features from the polygraph recordings, such as amplitude of the electrodermal response, increases in blood pressure, and respiratory suppression. Such features are known to reliably discriminate between truthful and deceptive people. A different set of features may be extracted from the signals collected during the oculomotor deception test that also discriminate between truthful and deceptive people. Likewise, oculomotor measures may be extracted from the polygraph phase of the test and traditional polygraph measures may be extracted from the ODT phase of the test.

The extracted features may be used to develop a separate logistic regression equation for each test. Results from the two tests then may be combined by means of a mathematical equation to compute an overall probability of deception. Alternatively, a single logistic regression equation may be developed that uses features from both tests to derive a single probability of deception. In either case, the final probability of deception may be compared to preestablished cutoffs to classify the test outcome as credible or deceptive. In some applications, two cutoffs may be used to establish an inconclusive region. For example, a logistic regression equation may weigh and combine the various features and produces the probability of deception. The results may then be subtracted from 1 to get the probability of truthfulness. Then the probability may be multiplied by 100 to determine a credibility score that ranges from 1 to 99. Depending on the reason for the test the cutoffs may be altered. For instance, in screening an applicant for a job a score greater than or equal to 50 it may be determined that the subject is credible. Alternatively a credibility test may be required for a criminal investigation. The parameters may be set such that if the score falls between 40 and 60, the outcome is inconclusive. The one or more cutoffs may be adjusted to maximize accuracy while minimizing inconclusive outcomes. These changes may be made in accordance with the intended purpose by a user.

In some embodiments of a hybrid test, during the polygraph portion four relevant issues of interest (R1, R2, R3, and R4) may be examined. Questions directed to these issues may be arranged in all possible pairs of the relevant issues. Two sessions of questions directed to polygraph type measures may be presented. In the first half of a first session, questions about the first relevant issue (R1) may be paired with questions about the second relevant issue (R2). In a second half of the first session, questions about the third relevant issue (R3) may be paired with questions about the fourth relevant issue (R4). In subsequent sessions, the first relevant issue (R1) may be paired with the third relevant issue (R3), the first relevant issue (R1) may be paired with the fourth relevant issue (R4), the second relevant issue (R2) may be paired with the third relevant issue (R3), and the second relevant issue (R2) may be paired with the fourth relevant issue (R4). During any given sub-session, the questions focus on only two of the four relevant issues. A separate session presenting questions directed towards oculomotor measure may be presented as a second phase of the test. Oculomotor, respiratory, electrodermal, cardiovascular, and somatic activity are recorded continuously throughout both phases of the hybrid test. The computer extracts features from the polygraph recordings, such as amplitude of the electrodermal response, increases in blood pressure, and respiratory suppression. Such features are known to reliably discriminate between truthful and deceptive people. A different set of features may be extracted from the signals collected during the oculomotor deception test that also discriminate between truthful and deceptive people. Likewise, oculomotor measures may be extracted from the polygraph phase of the test and traditional polygraph measures may be extracted from the ODT phase of the test

In another exemplary protocol, the system may administer a hybrid test that measures a test subject's cognitive and emotional reactions to test questions during a single phase that optimizes the diagnostic value of traditional polygraph measures while capturing oculomotor measures.

Test questions may be presented aurally and/or onscreen. In some embodiments, the presentation style of the questions may be mixed to avoid habituation. Questions may be answered “Yes” or “No” with a keypress or mouse click, and may be spaced 20 seconds or more apart to give physiological reactions sufficient time to recover before the next question is presented. Thus, test questions may be presented at a rate of two or three questions a minute. In contrast, the oculomotor test questions are presented at a faster pace. Oculomotor, respiratory, electrodermal, cardiovascular, and somatic activity are recorded continuously throughout the test. The computer extracts features from the polygraph recordings, such as amplitude of the electrodermal response, increases in blood pressure, and respiratory suppression. Such features are known to reliably discriminate between truthful and deceptive people. A different set of features may be extracted from the oculomotor measurements that also discriminate between truthful and deceptive people.

The extracted features may be used to develop a separate logistic regression equation for type of measurement during the test. Results from the two regressions may be combined by means of a mathematical equation to compute an overall probability of deception. Alternatively, a single logistic regression equation may be developed that uses features from both measurements to derive a single probability of deception. In either case, the final probability of deception may be compared to preestablished cutoffs to classify the test outcome as credible or deceptive. In some applications, two cutoffs may be used to establish an inconclusive region.

Example testing protocols in accordance with the present disclosure may use the testing procedures currently used for deception detection with oculomotor measurements, including those disclosed in US 2010/0324454 and Applicant's co-pending U.S. provisional patent application Ser. No. 63/033,600, filed Jun. 2, 2020 and entitled OCULOMOTOR BASED DECEPTION DETECTION USING AUDIO MULTI-ISSUE COMPARISON TESTING, the contents of which are incorporated by reference herein as if set forth in their entirety, and others. Such protocols may include the multi-issue comparison test (MCT), the audio multi-issue comparison test (AMCT), the relevant comparison test (RCT), and the directed lie comparison test (DLC) protocols which are commercially available from CONVERUS, among others. As a testing procedure is performed, oculomotor data are collected from the test subject and may be stored and processed as discussed therein to generate the probability of deception or its complement, a credibility score. It is noted that with oculomotor data alone, the MCT protocol has shown to be 88% accurate at correctly categorizing individuals' responses with respect to true positives (TP) and true negatives (TN), on specific testing issues in a laboratory study conducted by Dr. Andrew Potts at the University of Utah in 2019.

As discussed previously herein, although accuracy rates for known polygraph techniques can in some cases exceed 85 or 90 percent, they depend on the expertise and professionalism of the polygraph examiner who conducts and manually scores that test. Polygraph examiners, like all people, have biases that can affect how they interact with the test subject and how they evaluate the physiological recordings to reach a decision. Confrontational, awkward, sexually inappropriate, or racially insensitive pretest interactions with the test subject can adversely affect the subject, how they react to test questions, and the accuracy of the test outcome. In addition to interpersonal effects on polygraph accuracy, accuracy also depends on polygraph examiner's ability to interpret and score the physiological recordings. Typically, the polygraph examiner scores the test subject's reactions to test question after the test. Each channel of the polygraph is scored individually. For any channel, if the reaction to the comparison question is larger than the reaction to the relevant question, the score is from +1 to +3, depending on the magnitude of the difference. If the relevant reaction is larger, the score ranges from −1 to −3. The examiner assigns a score of 0 if there is little or no difference between reactions to relevant and comparison questions. The scores are summed over all channels and all repetitions of the questions to calculate subtotal and grand total scores. If the final score is sufficiently large and positive, then the subject is considered truthful. If the final score is sufficiently large and negative, then the subject is considered deceptive. If the result is close to zero, then the test is inconclusive. Thus, there is subjectivity in the manual scoring of polygraph recordings, and there can be considerable variance among different examiners who score the same recordings.

The hybrid systems and protocols in accordance with the present disclosure remove uncontrolled sources of bias and subjectivity. The computer enabled system administers and scores the test. Unlike a polygraph examiner, it is completely reliable and unbiased. In addition, features extracted by the computer from the polygraph and eye tracker recordings may be optimally weighed and combined by means of a logistic regression equation to compute the probability of credibility or deception. The system may produce a combined score for the entire test, as well as separate scores for the two subtests using combinations of the oculomotor, physiological, and behavioral data (response errors and response times). In a simple form, the system may produce a report similar to that depicted in FIG. 5.

Another immediate advantage is that systems and methods that include both oculomotor and traditional polygraph measures in accordance with the present disclosure may allow oculomotor-based deception detection to be used by organizations where local statutes restrict the use of lie detectors, other than an instrument that captures, measures and uses the data recorded by a traditional polygraph.

In some embodiments, tests performed using systems in accordance with the present disclosure can produce reliable determinations using only the “best data” available from the measurements taken during a testing procedure. For example, if physiological data are of poor quality or are missing due to movement artifacts, those data could be omitted from analysis and scoring. The system may report deception scores for polygraph physiological data and the oculomotor test data separately, and/or a final calculated score using all data, as may be desired or required for a particular user or setting. By using AI and machine learning techniques, the analysis of such data can be improved when sufficient data are accumulated and archived in a database.

While this disclosure has been described using certain embodiments, it can be further modified while keeping within its spirit and scope. This application is therefore intended to cover any variations, uses, or adaptations of the disclosure using its general principles. This application is intended to cover any and all such departures from the present disclosure as come within known or customary practices in the art to which it pertains, and which fall within the limits of the appended claims.

What is claimed is:

1. A system for deception detection assessment and credibility analysis including:
a processor, the processor presenting to a subject one or more questions in an assessment protocol;
an eye tracking device connected to the processor comprising:
an infrared camera that is positioned to be able to track an oculomotor movement of the subject during the assessment protocol;
the processor extracting features from oculomotor data obtained by the eye-tracking device to calculate a cognitive load assessment from the assessment protocol;
a set of physiological sensors in operative communication with the processor. comprising:
an electrodermal sensor connected to the processor positioned to be able to detect the electrodermal activity of the subject during the assessment protocol;
a respiration sensor connected to the processor positioned to be able to detect respiration of the subject during the assessment protocol;
at least one cardiovascular sensor connected to the processor positioned to be able to detect the cardiovascular activity of the subject during the assessment protocol;
the processor extracting features from physiological data obtained by the set of physiological sensors during the assessment protocol to calculate a first probability of deception;
the processor computing an overall probability of deception from the cognitive load assessment and the first probability of deception.

2. The system for deception detection assessment analysis of claim 1, wherein the processor is presenting to the subject one or more questions to obtain an emotional assessment and extracting features from oculomotor data obtained by the eye-tracking device to calculate an emotional assessment from the assessment protocol.

3. The system for deception detection assessment analysis of claim 2, further comprising the processor calculating computing an overall probability of deception from the emotional assessment, the cognitive load assessment, and the first probability of deception.

4. The system for deception detection assessment analysis of claim 1, wherein the assessment protocol features questions directed to four issues of interest presented in a series of session, with only questions directed to two of the four issues of interest presented in each session.

5. The system for deception detection assessment analysis of claim 1, further comprising a speaker connected to the processor positioned to be able to transmit sound to the subject; and the wherein the processor presents one or more questions to the subject aurally.

6. The deception detections system of claim 1, wherein the overall probability of deception is calculated by comparing data collected from the subject to preestablished cutoffs to classify the assessment outcomes as credible or deceptive.

7. The deception detection system of claim 1, wherein the processor presents questions to the subjects with a plurality of different time intervals between questions.

8. The deception detection system of claim 1, wherein the camera further positioned to be able to detect the pupil size of the subject during the assessment protocol.

9. The deception detection system of claim 1, wherein the at least one cardiovascular sensor detects pulse transit time of the subject during the assessment protocol.

10. The deception detections system of claim 1, wherein set of physiological sensors further comprises a body movement sensor connected to the processor positioned to be able to track body movements of the subject during the assessment protocol.

11. A process for credibility analysis comprising:
transmitting, by one or more processors, a series of questions to a subject as a credibility assessment;
receiving, by the one or more processors, from an eye tracking device that includes an infrared camera, data regarding the oculomotor movement of the subject during the credibility assessment;
receiving, by the one or more processors, from a set of physiological sensors, data regarding physiological responses of the subject during the credibility assessment, wherein the set of physiological sensors comprises at least one cardiovascular sensor, a respiration sensor, and an electrodermal sensor;
calculating, by the one or more processors, a cognitive load of the subject using the collected oculomotor movement data received by the processor from the eye tracking device;
determining, by the one or more processors, a first credibility score of the subject using the collected physiological data received by the processor from the set of physiological sensors; and
computing an overall probability of deception from the calculated cognitive load and the first probability of deception.

12. The process according to claim 11, further comprising receiving, by the one or more processors, a pupil size of a subject from the eye tracking device during the credibility assessment.

13. The process according to claim 11, wherein the processor is presenting to the subject one or more questions to obtain an emotional assessment., and calculating, by the one or more processors, an emotional assessment using the physiological measurements of the subject.

14. The process according to claim 13, wherein computing an overall probability of deception from the calculated cognitive load and the first probability of deception further comprises computing an overall probability of deception from the emotional assessment, the calculated cognitive load, and the first probability of deception.

15. The process according to claim 11, wherein transmitting, by one or more processors, the series of questions to a subject as a credibility assessment comprises presenting questions to the subject with a plurality of different time intervals between questions.

16. The process according to claim 11, wherein transmitting, by one or more processors, the series of questions to a subject as a credibility assessment comprises transmitting the series of questions to the subject aurally.

17. The process according to claim 11, wherein the overall probability of deception is calculated by comparing data collected from the subject to preestablished cutoffs to classify the assessment outcomes as credible or deceptive.

18. The process according to claim 11, wherein transmitting, by one or more processors, the series of questions to a subject as a credibility assessment comprises presenting questions to the subject directed to four issues of interest presented in a series of sessions, with only questions directed to two of the four issues of interest presented in each session.

19. The process according to claim 11, wherein the cardiovascular sensor detects pulse transit time of the subject during the assessment.

20. The process according to claim 11, wherein receiving, by the one or more processors, from a set of physiological sensors, data regarding physiological responses of the subject during the credibility assessment further receiving data from a body movement sensor tracking body movements of the subject during the assessment protocol.
